(19) United States
(12) Patent Application Publication
(10) Pub. No.: US 2021/0277352 A1
(43) Pub. Date: Sep. 9, 2021

(54) ENRICHMENT SUPPLEMENT FOR ANTIMICROBAL MATRICES

(71) Applicant: 3M INNOVATIVE PROPERTIES COMPANY, St. Paul, MN (US)

(72) Inventors: Gregory W. Sitton, Minneapolis, MN (US); Wilfredo Dominguez-Nunez, Minneapolis, MN (US)

(21) Appl. No.: 17/252,401

(22) PCT Filed: Jun. 13, 2019
(86) PCT No.: PCT/IB2019/054966

§ 371 Date: Dec. 15, 2020

Related U.S. Application Data
(60) Provisional application No. 62/687,449, filed on Jun. 20, 2018.

(51) Int. Cl. C12N 1/20

(52) U.S. Cl. CPC C12N 1/20

(57) ABSTRACT

Salts of Fe (II), Zn (II), or both used in methods for enriching food matrices containing antimicrobial compounds. A kit comprising salts of Fe (II), Zn (II) useful for such methods.

### BACKGROUND

In a process of culturing microorganisms from a particular source, such as a food matrix, the matrix is processed and then mixed with an enrichment media to facilitate the growth of the microorganism of interest. Enrichment media can be general enrichment media that is designed to facilitate growth of a variety of microorganisms, or specific enrichment media that is designed to facilitate growth of only desired microorganisms. The enriched matrix can then be inoculated on a culture plate and the presence or absence of the microorganism detected.

### SUMMARY

A method of growing of a target microorganism can comprise incubating a matrix having one or more antimicrobial compounds that are active against the target microorganism in the presence of an Fe(II) salt or a Zn(II) salt. A kit, such as a kit for carrying out this method, can comprise enrichment media that is selective for Salmonella or Listeria; and a Zn (II) salt or an Fe (II) salt.

### DETAILED DESCRIPTION

Throughout this disclosure, singular forms such as “a,” “an,” and “the” are often used for convenience; however, the singular forms are meant to include the plural unless the singular alone is explicitly specified or is clearly indicated by the context. When the singular alone is called for, the term “one and only one” is typically used.

Some terms in this disclosure are defined below. Other terms will be familiar to the person of skill in the art, and should be afforded the meaning that a person of ordinary skill in the art would have ascribed to them.

The terms “common,” “typical,” and “usual,” as well as “commonly,” “typically,” and “usually” are used herein to refer to features that are often employed in the invention and, unless specifically used with reference to the prior art, are not intended to mean that the features are present in the prior art, much less that those features are common, usual, or typical in the prior art.

Sample matrixes, such as food matrixes, may have very low levels of undesirable microorganisms. In order to ensure that the presence or absence of such microorganisms can be correctly confirmed, a matrix can be enriched before being cultured. Enrichment is typically accomplished by way of dispersing or dissolving the matrix in an enrichment medium, which can be added to a culture broth. There are two general categories of enrichment media. The first, general enrichment media, facilitates the growth of a variety of microorganisms. The second, selective enrichment media, facilitates the growth of one or more target microorganisms at the expense of at least one other, non-target microorganism.

Some matrixes have antimicrobial compounds that inhibit the growth of microorganisms, for example, they may inhibit the growth of certain target microorganisms. As an example, both nut and chocolate matrices may contain polyphenol compounds that exhibit antibiotic properties, as may coffee, tea, fruit, and vegetable matrices. Because of this a large amount of enrichment media is required to enrich matrices having antibiotic compounds, and particularly those with polyphenol antibiotic compounds such as nuts and chocolate, making enrichment and detection costly.

The inventors have identified several technical problems related to this issue. One problem is how to decrease the cost of enriching microorganisms in matrices having antimicrobial compounds. Defined slightly differently, this problem relates to decreasing the cost of enriching matrices having polyphenol antimicrobial compounds. Defined slightly differently, the problem relates to decreasing the cost of enriching Listeria or Salmonella in matrices having antimicrobial compounds that are active against Listeria or Salmonella. Such matrices can be, for example, nut or chocolate matrices, and the antimicrobial compounds can be polyphenols. A related problem is how to decrease the amount of enrichment media required for enriching microorganisms in matrices having antimicrobial compounds. Defined slightly differently, this problem relates to reducing the amount of enrichment media needed when enriching matrices having polyphenol antimicrobial compounds. Defined slightly differently, the problem relates to reducing the amount of enrichment media needed when enriching Listeria or Salmonella in matrices having antimicrobial compounds that are active against Listeria or Salmonella.

Briefly, the solution, which is described in more detail herein, lies in the use of Fe (II) or Zn (II) ion in the culture broth or enrichment media during the enrichment step. Surprisingly and unexpectedly, the addition of one or both of these ions increases the yield of microbes after enrichment, even without diluting the samples with high amounts of enrichment media. This eliminates the need for and cost of using high amounts of enrichment media in order to achieve satisfactory microorganism yield. No other ion that was tested, including Fe (III), showed this result.

Target microorganisms can be grown, or a matrix enriched in quantity of target microorganism, by incubating a matrix, such as a food matrix. While any matrix can be used, the methods described herein are most beneficial when used with matrices having one or more antimicrobial compounds that are active against the target microorganism. The matrix is incubated in the presence of an Fe (II) or Zn (II) salt, which provides Fe (II) or Zn (II) ions. By “Fe (II) or Zn (II)” it is meant that either Fe (II) or Zn (II) or both Fe (II) and Zn (II) can be present, although in most cases only one is required.

Most commonly the matrix is a food matrix, although other biological matrices, for example, blood, urine, mucous, saliva, feces, and the like, as well as environmental matrices such as soil, water, such as waste water, and the like can also be used. The most often used food matrices are nuts, such as peanuts, hazelnuts, cashews, or almonds, seeds, such as sunflower seeds or pumpkin seeds, dried fruits such as raisins, cranberries, or currents, and chocolate, such as milk chocolate, dark chocolate, white chocolate, or flavored chocolate. The matrix can comprise one or more of a peanut matrix, a sunflower seed matrix, and almond matrix, a cashew matrix, a raisin matrix, or a pumpkin seed matrix. In many cases, nuts or chocolate are the matrix. In some cases, the matrix is a nut matrix.

The antimicrobial compounds can be any antimicrobial compound, including pharmaceuticals such as sulfa drugs and penicillin or penicillin analogs or derivatives, but is most often polyphenol compounds. Such compounds can be naturally occurring in nuts or chocolate, among others.

The target microorganism can in principle be any microorganism. Most commonly, the target microorganism is Salmonella or Listeria. In most cases, the microorganism is neither a yeast nor an iron oxidizing or reducing microorganism.

The method can include a step admixing the matrix, a culture broth, and the Fe (II) or Zn (II) salt. The Fe(II) salt or Zn(II) salt can be used at any suitable concentration. A suitable concentration is one wherein the target microorganism can grow in sufficient yield without the need for unduly high amounts of enrichment media. Typically, the Fe (II) salt or Zn (II) salt is at a concentration of 1 mg/L or greater 5 mg/L or greater, 10 mg/L or greater, 25 mg/L or greater, 50 mg/L or greater, 75 mg/L or greater, 100 mg/L or greater, 250 mg/L or greater, 500 mg/L or greater, 750 mg/L or greater, 1,000 mg/L or greater, 1,250 mg/L or greater, 1,500 mg/L or greater 1,750 mg/L or greater, or 2,000 mg/L or greater. Commonly, the Fe (II) ion or the Zn (II) ion is at a concentration of 2,000 mg/L or less, 1,750 mg/L or less, 1,500 mg/L or less, 1,250 mg/L or less, 1,000 mg/L or less, 750 mg/L or less, 500 mg/L or less, 250 mg/L or less, 100 mg/L or less, 75 mg/L or less, 50 mg/L or less, 25 mg/L or less, 10 mg/L or less, or 5 mg/L or less.

The Fe (II) salt or Zn (II) salt, the matrix, and the culture broth can be admixed, wherein the Fe(II) salt or Zn(II) salt is at a concentration of 50 micromolar or greater, 100 micromolar or greater, 250 micromolar or greater, 300 micromolar or greater, 400 micromolar or greater, 500 micromolar or greater, 600 micromolar or greater, 700 micromolar or greater, 800 micromolar or greater, 900 micromolar or greater, or 1,000 micromolar or greater. The Fe (II) salt or Zn (II) salt, the matrix, and the culture broth can be admixed, wherein the Fe(II) salt or Zn(II) salt is at a concentration of 1,000 micromolar or less, 900 micromolar or less, 800 micromolar or less, 700 micromolar or less, 600 micromolar or less, 500 micromolar or less, 400 micromolar or less, 300 micromolar or less, 200 micromolar or less, or 100 micromolar or less.

In many cases, the admixing steps described herein include dissolving or dispersing the matrix in a culture broth containing an enrichment medium. The Fe (II) or Zn (II) salt or salts man be part of the enrichment medium, or can be added separately.

Any type of enrichment media can be used. In many cases, the enrichment media is a general enrichment medium that facilitates the growth of a wide variety of microorganisms without selecting for the target microorganism. More frequently, the enrichment medium is a selective enrichment medium that preferentially allows growth of the target microorganism at the expense of other microorganisms. Typical enrichment media can include, for example, salts, buffers, nutrients, and the like. An example of a general enrichment medium is Buffered Peptone Water that contains peptone, sodium chloride, disodium phosphate and monopotassium phosphate. An example of a selective enrichment medium is Demi Fraser broth (both available from 3M Company, St. Paul, Minn., USA) that contains enzymatic digest of animal origin, beef and yeast extract, sodium chloride, disodium phosphate, monopotassium phosphate, esculin, acriflavine, nalidixic acid and lithium chloride.

The Fe (II) or Zn (II) salts can be any salt that is effective to provide good yield of the target microorganism, particularly without using an unduly high amount of enrichment media. Most commonly, the Fe (II) or Zn (II) salts will be at least sparingly soluble in water, such as partially soluble in water or fully soluble in water. Exemplary Fe (II) salts include ferrous sulfate, ferrous chloride, ferrous bromide, ferrous iodide, ferrous phosphate, ferrous acetate, ferrous nitrate, ferrous citrate, ferrous tartrate, ferrous besylate, ferrous mesylate, ferrous carbonate, or ferrous tosylate. More commonly, the Fe (II) salt is in the form of ferrous sulfate, ferrous chloride, or ferrous citrate. Ferrous sulfate is most common. Exemplary Zn (II) salts include zinc sulfate, zinc chloride, zinc bromide, zinc iodide, zinc acetate, zinc nitrate, zinc citrate, zinc tartrate, zinc besylate, zinc mesylate, zinc carbonate, or zinc tosylate. More commonly, the Zn (II) salt is in the form of zinc sulfate, zinc chloride, or zinc acetate. Zinc chloride is most common.

Once the matrix, enrichment medium, and culture broth are admixed, the admixture can be allowed to incubate in order to enrich the matrix. The particular enrichment conditions, such as time and temperature, will depend on the target microortanism. Typical enrichment times vary from 1 hour to 48 hours, such as 1 hour to 36 hours, 1 hour to 24 hours, 1 hour to 12 hours, or even less. Typical enrichment temperatures are 20° C. to 40° C., such as 25° C. to 37° C. One common temperature is 37° C. Typically, enrichment is deemed to be sufficient if the target microorganism is recovered from the culture broth at a concentration of 10⁷ CFU/mL or higher. The amount of enrichment media required to do this is reduced, for example by 10% or more, 20% or more, 30% or more, 40% or more, or even 50% or more, as compared to an identical enrichment method that does not use Fe (II) or Zn (II) salts. In some cases, the target microorganism cannot be enriched to a concentration of 10⁷ CFU/mL or higher without the use of Fe (II) or Zn (II) salts.

The method can comprise a step of inoculating the matrix, or a solution or dispersion of the matrix, onto a culture plate. This can be done as part of enrichment of the matrix, or it can be done after the matrix is enriched. The culture plate can be an agar culture plate, such as those commonly known in the art. For example, Xylose Lysine Deoxycholate (XLD) Agar (available from Hardy Diagnostics, Santa Maria, Calif., USA) for Salmonella, and Modified Oxford (MOX) Agar (available under the REMEL trade designation, a brand of Thermo Fisher Scientific Waltham, Mass., USA)) for Listeria spp. The culture plate can also be a thin-film culture plate, such as the PETRIFILM™ brand of culture plates that are commercially available from 3M Company (St. Paul, Minn., USA).

The method can further comprise detecting the presence or absence of the target microorganism. After inoculation onto a culture plate, the culture plate can be further incubated and the presence or absence of the target microorganism can be detected. Detection can be accomplished by visual inspection, but is more often accomplished by use of a plate reader, such as the 3M Petrifilm™ Plate Reader (3M Company, St. Paul, Minn., USA).

A kit can be provided for performing the methods described herein. Most commonly, the kit will include an enrichment medium, and particularly an enrichment medium that is selective for Listeria or Salmonella, and an Fe (II) or Zn (II) salt. The Fe (II) or Zn (II) salt can be present as a component of the enrichment medium or separately from the enrichment medium.

The following exemplary embodiments are presented to illustrate particular aspects of the disclosure, and are not meant to be limiting. Other embodiments are also disclosed.

1. A method of growing of a target microorganism comprising incubating a matrix having one or more antimicrobial compounds that are active against a target microorganism in the presence of an Fe(II) salt or a Zn(II) salt.
2. The method of embodiment 1, wherein the matrix is a food matrix.
3. The method of any of embodiments 1 or 2, wherein the one or more antibicrobial compounds comprise one or more polyphenols 4. The method of any of the preceding embodiments, wherein the matrix comprises one or more of a nut matrix, a seed matrix, a dried fruit matrix, or a chocolate matrix.
5. The method of any of the preceding embodiments, wherein the matrix comprises a nut matrix.
6. The method of any of the preceding embodiments, wherein the matrix comprises a seed matrix.
7. The method of any of the preceding embodiment, wherein the matrix comprises a dried fruit matrix.
8. The method of any of the preceding embodiments, wherein the matrix comprises a chocolate matrix.
9. The method of any of embodiments 1-4, wherein the matrix comprises one or more of a peanut matrix, a sunflower seed matrix, and almond matrix, a cashew matrix, a raisin matrix, or a pumpkin seed matrix.
10. The method of any of the preceding embodiments, wherein the target microorganism is Listeria or Salmonella.
11. The method of any of the preceding embodiments, wherein the microorganism is neither a yeast nor an iron oxidizing or reducing microorganism.
12. The method of any of the preceding embodiments, further comprising admixing the matrix, a culture broth, and the Fe (II) or Zn (II) salt, wherein the Fe(II) salt or Zn(II) salt is at a concentration of 1 mg/L or greater 5 mg/L or greater, 10 mg/L or greater, 25 mg/L or greater, 50 mg/L or greater, 75 mg/L or greater, 100 mg/L or greater, 250 mg/L or greater, 500 mg/L or greater, 750 mg/L or greater, 1,000 mg/L or greater, 1,250 mg/L or greater, 1,500 mg/L or greater 1,750 mg/L or greater, or 2,000 mg/L or greater.
13. The method of any of the preceding embodiments, further comprising admixing the matrix, a culture broth, and the Fe (II) or Zn (II) salt, wherein the Fe(II) salt or Zn(II) salt is at a concentration of 2,000 mg/L or less, 1,750 mg/L or less, 1,500 mg/L or less, 1,250 mg/L or less, 1,000 mg/L or less, 750 mg/L or less, 500 mg/L or less, 250 mg/L or less, 100 mg/L or less, 75 mg/L or less, 50 mg/L or less, 25 mg/L or less, 10 mg/L or less, or 5 mg/L or less.
14. The method of any of the preceding embodiments, further comprising admixing the matrix, a culture broth, and the Fe (II) or Zn (II) salt, wherein the Fe(II) salt or Zn(II) salt is at a concentration of 50 micromolar or greater, 100 micromolar or greater, 250 micromolar or greater, 300 micromolar or greater, 400 micromolar or greater, 500 micromolar or greater, 600 micromolar or greater, 700 micromolar or greater, 800 micromolar or greater, 900 micromolar or greater, or 1,000 micromolar or greater.
15. The method of any of the preceding embodiments, further comprising admixing the matrix, a culture broth, and the Fe (II) or Zn (II) salt, wherein the Fe(II) salt or Zn(II) salt is at a concentration of 1,000 micromolar or less, 900 micromolar or less, 800 micromolar or less, 700 micromolar or less, 600 micromolar or less, 500 micromolar or less, 400 micromolar or less, 300 micromolar or less, 200 micromolar or less, or 100 micromolar or less.
16. The method of any of the preceding embodiments, further comprising dissolving or dispersing the matrix in a culture broth containing an enrichment medium.
17. The method of embodiment 12, wherein the enrichment medium is a general enrichment medium.
18. The method of embodiment 12, wherein the enrichment medium is selective for the target microorganism.
19. The method of any of the preceding embodiments, wherein the enrichment medium comprises salt.
20. The method of any of the preceding embodiments, wherein the enrichment medium comprises nutrients.
21. The method of any of the preceding embodiments, wherein the enrichment medium comprises buffer.
22. The method of any of the preceding embodiments, wherein the enrichment medium comprises 23. The method of any of the preceding embodiments, further comprising inoculating the matrix or a solution or suspension of the matrix onto a culture plate.
24. The method of embodiment 19, wherein the culture plate is an agar culture plate.
25. The method of embodiment 19, wherein the culture plate is a thin film culture plate.
26. The method of any of the preceding embodiments, wherein the Fe (II) salt or Zn (II) salt is at least sparingly soluble in water.
27. The method of any of the preceding embodiments, wherein the Fe (II) salt or Zn (II) salt is at least partially soluble in water.
28. The method of any of the preceding embodiments, wherein the Fe (II) salt or Zn (II) salt is freely soluble in water.
29. The method of any of the preceding embodiments, wherein the Fe (II) ion is in the form of ferrous sulfate, ferrous chloride, ferrous bromide, ferrous iodide, ferrous phosphate, ferrous acetate, ferrous nitrate, ferrous citrate, ferrous tartrate, ferrous besylate, ferrous mesylate, ferrous carbonate, or ferrous tosylate
30. The method of any of the preceding embodiments, wherein the Fe (II) ion is in the form of ferrous sulfate, ferrous chloride, or ferrous citrate.
31. The method of any of the preceding embodiments, wherein the Fe (II) ion is in the form of ferrous sulfate.
32. The method of any of the preceding embodiments, wherein the Zn (II) ion is in the form of zinc sulfate, zinc chloride, zinc bromide, zinc iodide, zinc acetate, zinc nitrate, zinc citrate, zinc tartrate, zinc besylate, zinc mesylate, zinc carbonate, or zinc tosylate.
33. The method of any of the preceding embodiments, wherein the Zn (II) ion is in the form of zinc sulfate, zinc chloride, or zinc acetate
34. The method of any of the preceding embodiments, wherein the Zn (II) ion is in the form of zinc chloride.
35. The method of any of the preceding embodiments, further comprising detecting the presence of a target microorganism.
36. A kit comprising:
enrichment media that is selective for Salmonella or Listeria; and
a Zn (II) salt or an Fe (II) salt.
37. The kit of embodiment 36, wherein the Zn (II) salt or an Fe (II) salt is a component of the enrichment media.
38. The kit of embodiment 37, wherein the Zn (II) salt or Fe (II) salt is separate from the enrichment media.

### EXAMPLES

### Materials

Ferrous sulfate (FeSO₄), zinc chloride (ZnCl₂), calcium chloride (CaCl₂)), magnesium sulfate (MgSO₄), nickel sulfate (NiSO₄), barium chloride (BaCl₂), ferric citrate (C₆H₅FeO₇), and ferric ammonium citrate (C₆H₁₁FeNO₇) were obtained from the Sigma-Aldrich Corporation, St. Louis, Mo.

Demi Fraser enrichment media was obtained from the 3M Company, St. Paul, Minn.

Tryptic soy broth (TSB) was obtained from Becton, Dickinson and Company, Franklin Lakes, N.J.

WHIRL-PAK sterile sampling bags were obtained from the Nasco Company, Fort Atkinson, Wis.

Buffered Peptone Water (BPW), Modified Oxford (MOX) agar plates, and EZ-CHROM Salmonella Plus agar plates were obtained from Microbiology International, Frederick, Md.

Salmonella typhimurium (ATCC 14028) and Listeria innocua (ATCC 33090) were obtained from Microbiologics Incorporated, St. Cloud, Minn.

Salmonella typhimurium (ATCC 14028) and Listeria innocua (ATCC 33090) were individually incubated overnight in tryptic soy broth at 37° C. for 18 hours. Individual inoculums were prepared by serially diluting each culture sample with Butterfield's Buffer (3M Company, St. Paul, Minn.). To determine the cell concentration (cfu/sample), a 1 mL aliquot of diluted sample was plated onto a PETRIFILM Aerobic Count Plate (3M Company, St. Paul, Minn.), incubated, and counted according to the manufacturer's instructions. The counts (cfu) from the plates were used to calculate the final dilution required to achieve the targeted inoculum concentration.

### Examples 1-3 and Comparative Example A

A food matrix containing 25 g of a trail mix blend (containing peanuts, raisins, sunflower seeds, almonds, cashews, pumpkin seeds, and vegetable oil) was inoculated with a low level [calculated as about 10 colony forming units (cfu)] of Salmonella typhimurium (ATCC 14028) by delivering 100 microliters of a diluted culture directly to the food matrix. The inoculated sample was added to a WHIRL-PAK sterile sampling bag that contained 100 mL of Buffered Peptone Water enrichment media. Next, ferrous sulfate heptahydrate was added to the media at a concentration of either 50 mg/L (Example 1), 500 mg/L (Example 2), 2000 mg/L (Example 3), or 0 mg/mL (Comparative Example A). The bag was closed and the sample was mixed for 30 seconds followed by incubation at 35° C. for 24 hours. A 10 mL aliquot of the media was removed and serially diluted with Demi Fraser media (3M Company, St. Paul, Minn.). A 100 microliter sample from the final dilution was plated onto an EZ-CHROM Salmonella Plus agar plate according to the manufacturer's instructions and the plate was incubated at 37° C. for 24 hours. Colonies (cfu) that formed were counted by visual inspection. The total colony count post enrichment was calculated by adjusting the observed plate count based on the number and volume of the dilutions. The recovery of Salmonella typhimurium (cfu/mL) after enrichment is reported in Table 1 as the mean value of two replicates.

TABLE 1
Recovery of Salmonella typhimurium from an Enriched Food Matrix

| | | BPW | | Initial Sample | Recovery after |
|---|---|---|---|---|---|
| | Food | Media | FeSO₄ | Inoculation | Enrichment |
| | Matrix (g) | (mL) | (mg/L) | (cfu/sample) | (cfu/mL) |
| Example 1 | 25 | 100 | 50 | 10 | 1 × 10⁶ |
| Example 2 | 25 | 100 | 500 | 10 | 1 × 10⁶ |
| Example 3 | 25 | 100 | 2000 | 10 | 1 × 10⁷ |
| Comparative | 25 | 100 | 0 | 10 | 1 × 10⁵ |
| Example A | | | | | |

### Examples 4-6 and Comparative Example B

A food matrix containing 25 g of a trail mix blend (containing peanuts, raisins, sunflower seeds, almonds, cashews, pumpkin seeds, and vegetable oil) was inoculated with a low level [calculated as about 1.4 colony forming units (cfu)] of Listeria innocua (ATCC 33090) by delivering 100 microliters of a diluted culture directly to the food matrix. The inoculated sample was added to a WHIRL-PAK sterile sampling bag that contained 100 mL of Demi Fraser enrichment media. Next, ferrous sulfate heptahydrate was added to the media at a concentration of either 50 mg/L (Example 4), 500 mg/L (Example 5), 2000 mg/L (Example 6), or 0 mg/L (Comparative Example B). The bag was closed and the sample was mixed for 30 seconds followed by incubation at 35° C. for 24 hours. A 10 mL aliquot of the media was removed and serially diluted with fresh Demi Fraser media. A 100 microliter sample from the final dilution was plated onto a Modified Oxford (MOX) agar plate according to the manufacturer's instructions and the plate was incubated at 37° C. for 24 hours. Colonies (cfu) that formed were counted by visual inspection. The total colony count post enrichment was calculated by adjusting the observed plate count based on the number and volume of the dilutions. The recovery of Listeria innocua (cfu/mL) after enrichment is reported in Table 2 as the mean value of two replicates.

TABLE 2
Recovery of Listeria innocua from an Enriched Food Matrix

| | | Demi | | | |
|---|---|---|---|---|---|
| | Food | Fraser | | Initial Sample | Recovery after |
| | Matrix | Media | FeSO₄ | Inoculation | Enrichment |
| | (g) | (mL) | (mg/L) | (cfu/sample) | (cfu/mL) |
| Example 4 | 25 | 100 | 50 | 1.4 | 1 × 10⁵ |
| Example 5 | 25 | 100 | 500 | 1.4 | 1 × 10³ |
| Example 6 | 25 | 100 | 2000 | 1.4 | 1 × 10³ |
| Comparative | 25 | 100 | 0 | 1.4 | 1 × 10⁴ |
| Example B | | | | | |

### Example 7 and Comparative Example C

A food matrix containing 50 g of a trail mix blend (containing peanuts, raisins, sunflower seeds, almonds, cashews, pumpkin seeds, and vegetable oil) was inoculated with a low level [calculated as about 3.7 colony forming units (cfu)] of Listeria innocua (ATCC 33090) by delivering 100 microliters of a diluted culture directly to the food matrix. The inoculated sample was added to a WHIRL-PAK sterile sampling bag that contained 200 mL of Demi Fraser enrichment media. Next, zinc chloride was added to the media at a concentration of either 45 mg/L (330 micromolar—Example 7) or 0 mg/L (control—Comparative Example C). The bag was closed and the sample was mixed for 30 seconds followed by incubation at 35° C. for 24 hours. A 100 microliter aliquot of the media was removed and serially diluted with fresh Demi Fraser media. A 100 microliter sample from the final dilution was plated onto a Modified Oxford (MOX) agar plate according to the manufacturer's instructions and the plate was incubated at 37° C. for 24 hours. Colonies (cfu) that formed were counted by visual inspection. The total colony count post enrichment was calculated by adjusting the observed plate count based on the number and volume of the dilutions. The recovery of Listeria innocua (cfu/mL) after enrichment is reported in Table 3 (and repeated in Table 4) as the mean value of two replicates.

TABLE 3
Recovery of Listeria innocua from an Enriched Food Matrix

| | | Demi | | | |
|---|---|---|---|---|---|
| | Food | Fraser | | Initial Sample | Recovery after |
| | Matrix | Media | ZnCl2 | Inoculation | Enrichment |
| | (g) | (mL) | (mg/L) | (cfu/sample) | (cfu/mL) |
| Example 7 | 50 | 200 | 45 | 3.7 | 1 × 10⁶ |
| Comparative | 50 | 200 | 0 | 3.7 | 1 × 10⁵ |
| Example C | | | | | |

### Comparative Example D

The same procedure as described in Example 7 was followed (50 g of trail mix blend in 200 mL of Demi Fraser media) with the exception that the ZnCl₂ (45 mg/L, 330 micromolar) was replaced with anhydrous CaCl₂ (36.5 mg/L, 330 micromolar). The recovery of Listeria innocua (cfu/mL) after enrichment is reported in Table 4 as the mean value of two replicates.

### Comparative Example E

The same procedure as described in Example 7 was followed (50 g of trail mix blend in 200 mL of Demi Fraser media) with the exception that the ZnCl₂ (45 mg/L, 330 micromolar) was replaced with anhydrous MgSO₄ (40 mg/L, 332 micromolar). The recovery of Listeria innocua (cfu/mL) after enrichment is reported in Table 4 as the mean value of two replicates.

### Comparative Example F

The same procedure as described in Example 7 was followed (50 g of trail mix blend in 200 mL of Demi Fraser media) with the exception that the ZnCl₂ (45 mg/L, 330 micromolar) was replaced with ferric citrate (81 mg/L, 331 micromolar). The recovery of Listeria innocua (cfu/mL) after enrichment is reported in Table 4 as the mean value of two replicates.

### Comparative Example G

The same procedure as described in Example 7 was followed (50 g of trail mix blend in 200 mL of Demi Fraser media) with the exception that the ZnCl₂ (45 mg/L, 330 micromolar) was replaced with ferric ammonium citrate (87.5 mg/L, 330 micromolar). The recovery of Listeria innocua (cfu/mL) after enrichment is reported in Table 4 as the mean value of two replicates.

### Comparative Example H

The same procedure as described in Example 7 was followed (50 g of trail mix blend in 200 mL of Demi Fraser media) with the exception that the ZnCl₂ (45 mg/L, 330 micromolar) was replaced with NiSO₄ hexahydrate (87 mg/L, 330 micromolar). The recovery of Listeria innocua (cfu/mL) after enrichment is reported in Table 4 as the mean value of two replicates.

### Comparative Example I

The same procedure as described in Example 7 was followed (50 g of trail mix blend in 200 mL of Demi Fraser media) with the exception that the ZnCl₂ (45 mg/L, 330 micromolar) was replaced with BaCl₂ dihydrate (81 mg/L, 331 micromolar). The recovery of Listeria innocua (cfu/mL) after enrichment is reported in Table 4 as the mean value of two replicates.

### Comparative Example J

The same procedure as described in Example 7 was followed (50 g of trail mix blend in 200 mL of Demi Fraser media) with the exception that the ZnCl₂ (45 mg/L, 330 micromolar) was not used. The recovery of Listeria innocua (cfu/mL) after enrichment is reported in Table 4 as the mean value of two replicates.

TABLE 4
Recovery of Listeria innocua from an Enriched Food Matrix

| | | Conc. of | | |
|---|---|---|---|---|
| | | Salt in | Initial | Recovery |
| | Salt added to | Enrichment | Sample | after |
| | Enrichment | Media | Inoculation | Enrichment |
| | Media | (micromolar) | (cfu/sample) | (cfu/mL) |
| Example 7 | ZnCl₂ | 330 | 3.7 | 1 × 10⁶ |
| Comparative | CaCl₂ | 330 | 3.7 | 1 × 10⁴ |
| Example D | | | | |
| Comparative | MgSO₄ | 332 | 3.7 | 1 × 10⁵ |
| Example E | | | | |
| Comparative | C₆H₅FeO₇ | 331 | 3.7 | 1 × 10⁴ |
| Example F | (Fe(III) citrate) | | | |
| Comparative | C₆H₁₁FeNO₇ | 330 | 3.7 | 1 × 10⁴ |
| Example G | Fe(III) | | | |
| | ammonium | | | |
| | citrate | | | |
| Comparative | NiSO₄ | 330 | 3.7 | 1 × 10⁵ |
| Example H | | | | |
| Comparative | BaCl₂ | 331 | 3.7 | 1 × 10⁴ |
| Example I | | | | |
| Comparative | none | 0 | 3.7 | 1 × 10⁵ |
| Example J | | | | |

1. A method of growing of a target microorganism comprising incubating a matrix having one or more antimicrobial compounds that are active against the target microorganism in the presence of an Fe(II) salt or a Zn(II) salt.

2. The method of claim 1, wherein the matrix is a food matrix.

3. The method of claim 1, wherein the matrix comprises a nut matrix, a food matrix, or a combination thereof.

4. The method of claim 1, wherein the target microorganism is Listeria or Salmonella.

5. The method of claim 1, wherein the microorganism is neither a yeast nor an iron oxidizing or reducing microorganism.

6. The method of claim 1, further comprising dissolving or dispersing the matrix in a culture broth containing an enrichment medium.

7. The method of any of the claim 6, further comprising admixing the matrix, the culture broth, and the Fe (II) or Zn (II) salt, wherein the Fe(II) salt or Zn(II) salt is at a concentration of 100 micromolar or greater.

8. The method of any of the claim 6, further comprising admixing the matrix, the culture broth, and the Fe (II) or Zn (II) salt, wherein the Fe(II) salt or Zn(II) salt is at a concentration of 1,000 micromolar or less.

9. The method of claim 7, wherein the enrichment medium is a selective enrichment medium for the target microorganism.

10. The method of claim 1, wherein the Zn (II) salt is in the form of zinc chloride.

11. The method of claim 1, wherein the Fe (II) salt is in the form of ferrous sulfate.

12. The method of claim 1, further comprising detecting the presence of a target microorganism.

13. A kit comprising:
enrichment media that is selective for Salmonella or Listeria; and
a Zn (II) salt or an Fe (II) salt.

14. The kit of claim 13, wherein the Zn (II) salt or an Fe (II) salt is a component of the enrichment media.

15. The kit of claim 13, wherein the Zn (II) salt or Fe (II) salt is separate from the enrichment media.

16. The method of claim 8, wherein the enrichment medium is a selective enrichment medium for the target microorganism.

17. The method of claim 4, wherein the enrichment medium is a selective enrichment medium for the target microorganism.
